(19) United States
(12) Patent Application Publication
(10) Pub. No.: US 2008/0090885 A1
(43) Pub. Date: Apr. 17, 2008

(54) Preparation of losartan 5-carboxylic acid and use thereof

(76) Inventors: Liqin Chen, Jiangsu (CN); Jian Li, Jiangsu (CN); Yingzhong Shen, Jiangsu (CN)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/699,703

(22) Filed: Jan. 29, 2007

(30) Foreign Application Priority Data
Oct. 12, 2006 (CN) 200610096726.5

(51) Int. Cl. A61K 31/41; C07D 257/00

(52) U.S. Cl. 514/381; 548/253

(57) ABSTRACT

A method for synthesis of losartan 5-carboxylic acid (EXP-3174).

### RELATED APPLICATION

The present application claims priority to Chinese Application No. 200610096726.5 filed Oct. 12, 2006, the disclosure of which is incorporated herein in its entirety by reference.

### BACKGROUND OF THE INVENTION

Losartan is a well known angiotension II receptor antagonist and with a chemical structure as shown in Formula I. It has an oral bioavailability of about 33% and is rapidly absorbed (peak plasma levels in 1 hour). Losartan itself is a potent, competitive AT₁ receptor antagonist; however it has a short half-life (about 2 hours). The effectiveness of a once-a-day dosing regimen is explained by the fact that a discrete percentage of an oral dose of losartan in human beings is converted to a 5-carboxylic acid metabolite, known as EXP-3174 as shown in Formula II, which is a non-competitive AT₁ receptor antagonist that is 10-40 times more potent than losartan and has a much longer half-life (6-9 hours) than losartan.

Because of the importance of the EXP-3174 in the mechanism of action of the losartan because the methods for the preparation of this compound do not appear to offer the possibility to be scale up, a new synthetic method for production of EXP-3174 is needed.

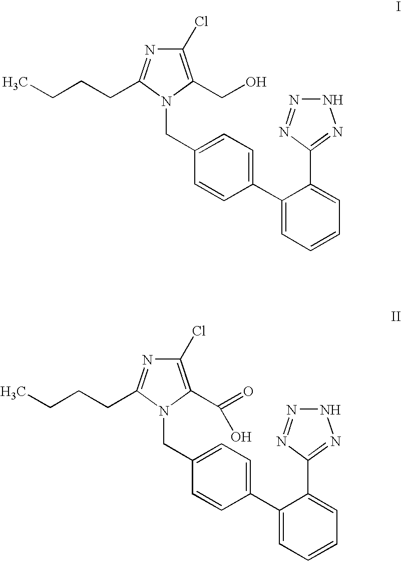

### DETAILED DESCRIPTION OF THE INVENTION

EXP-3174 was first synthesized in accordance with a method disclosed U.S. Pat. No. 5,138,069, the disclosure of which is hereby incorporated by reference in its entirety. The method involved the purification of the compound using flash column chromatography. A recent method (Tetrahedron Letters 44(2003) 1149-1152) reported the synthesis of this compound using microwave-assisted synthesis. This technique, however, sets definite limits on the scale of the reaction. Furthermore, EXP-3174 has been purified by preparative RP-HPLC.

The method of the present invention employs conventional synthetic methodology. Losartan or its salts were used as starting material. The compound was oxidized using common oxidation agent. The product was isolated by precipitation and purified by recrystallization. The product can easily reach required pharmaceutical purity with reasonable yield (over 70%).

The various inorganic and organic salts of losartan and EXP-3174 are also within the scope of the invention. Such salts include ammonium salts, alkali metal salts such as sodium and potassium salts, alkaline earth metal salts such as calcium and magnesium salts, salts with organic bases; e.g., dicyclohexylamine salts, N-methyl-D-glucamine salts, salts with amino acids such as arginine, lysine, and the like. Also, salts with organic and inorganic acids may be prepared; e.g., HCl, HBr, H₂SO₄, H₃PO₄, methanesulfonic acid, toluenesulfonic acid, maleic acid, fumaric acid, camphorsulfonic acid. The non-toxic, physiologically, acceptable salts are preferred, although other salts are also useful; e.g., in isolation or purification of the product.

The salts can be formed by conventional means such as by reacting the free acid or free base forms of the product with one or more equivalents of the appropriate base or acid in a solvent or medium in which the salt is insoluble, or in a solvent such as water which is then removed in vacuum or by freeze-drying or by exchanging the cations of an existing salt for another cation on a suitable ion exchange resin.

The compound of this invention is useful in treating hypertension. It is also of value in the management of acute and chronic congestive heart failure and angina. It is also expected to be useful in primary and secondary hyperaldosteronism, renal diseases such as diabetic nephritic syndrome, hypertensive nephrosclerosis, end stage renal disease, renal transplanttherapy, renovascular hypertension, scleroderma, left ventricular dysfunction, systolic and diastolic dysfunction diabetic retinopathy, in the management of vascular disorders such as migraine or Raynaud's disease, as prophylaxis to minimize the atherosclerotic process, in neointimal hyperplasia following angioplasty or vascular injury and to retard the onset of type II diabetes. The application of the compound this invention for these and similar disorder will be apparent to those skilled in the art.

The compound of this invention is also useful to treat elevated intraocular pressure and to enhance retinal blood flow and can be administered to patients in need of such treatment with typical pharmaceutical formulations such as tablets, capsules, injectables and the like as well as topical ocular formulation in the form of solution, ointments, inserts, gels, and the like. Pharmaceutical formulations prepared to treat intraocular pressure would typically contain about 0.1%, to 15% by weight, preferably 0.5% to 2% by weight, of the compound of this invention. For this use, the compound of this invention may also be used in combination with other medications for the treatment.

The compound of this invention can also be administered in combination with other antihypertensive and/or diuretics. For example, the compound of this invention can be given in combination with diuretics such as hydrochlorothiazide, chlorothiazide, chlorthalidone, methylclothiazide, furosemide, ethacrynic acid, triamteren, amiloride, atriopeptin and spironolactone; calcium channel blockers, such as diltiazem, felodipine, nifedipine, amlodipine, nimodipine, isradipine, nitrendipine and verapamil; adrenergic antagonists such as timolol, atenolol, metoprolol, propranolol, nadolol and pindolol; angiotensin converting enzyme inhibitors such as enalapril, lisinopril, captopril, ramipril, quinapril and zofenopril; rennin inhibitors such prazosin, doxazocin, and terazosin; sympatholytic agents such as methyldopa, clonidine and guanabenz, atriopeptidase inhibitors such as UK-79300; serotonin antagonists such as ketanserin; A2-adenosine receptor agonists such as CGS 22492; potassium channel agonists such as pinacidil and cromakalim; and various other antihypertensive drugs including reserpine, minoxidil, guanethidine, hydrazine hydrochloride and sodium nitroprusside as well as combinations of the above-named drugs.

Combinations useful in the management of congestive heart failure include, in addition, the compound of this invention with cardiac stimulants such as dobutamine and xamoterol and phosphodiesterase inhibitors including amrinone and milrinone.

Typically, the individual daily dosage for these combinations can range from about one-fifth of the minimally recommended clinical dosages to the maximum recommended levels for the entities when they are given singly.

Typically about 1 to 100 mg of the compound of Formula II or a physiologically acceptable salt thereof is compounded with a physiologically acceptable vehicle, carrier, excipient, binder, preservative, stabilizer, flavor, etc., in a unit dosage form as called for by accepted pharmaceutical practice. The amount of active substance in these compositions or preparations is such that a suitable dosage in the range indicated is obtained.

Illustrative of the adjuvants which can be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; a flavoring agent such as peppermint, oil of wintergreen or cherry. When the dosage unit form is a capsule, it may contain, in addition to material of the above type, a liquid carrier such as fatty oil. Various other materials may be present as coatings or to otherwise modify the physical form of the dosage unit.

Sterile compositions for injection can be formulated according to conventional pharmaceutical practice by dissolving or suspending the active substance in a vehicle such as water for injection, a naturally occurring vegetable oil like sesame oil, coconut oil, peanut oil, cottonseed oil, etc., or a synthetic fatty vehicle like ethyl oleate or the like. Buffers, preservatives, antioxidants and the like can be incorporated as required.

### Synthesis

To a 2000 ml three necked round bottom flask equipped with condenser, mechanical stirrer, and thermometer was added de-ionized water (250 ml), potassium permanganate (0.25 mol) and stirred for about 15 minutes. Tetrabutylammonium chloride (0.30 ml) was added in four portions in 20 minutes. After stirring vigorously for 30 minutes, pyridine (100 ml) and acetone (650 ml) were added and the reaction mixture stirred for another 30 minutes.

The dark purple solution was transferred into a 2000 ml three necked flask equipped with condenser, mechanical stirrer, and thermometer. The solution was warmed up to an internal temperature 40° C. Losartan (0.1 ml) [QUESTION: What is the total amount by weight of Losartan?] was added into the reaction mixture in portions with continued stirring. The internal temperature of the reaction mixture was maintained below 50° C. After one hour, the reaction was completed. To the reaction mixture, was added 30% formaldehyde solution (200 ml) slowly with the temperature maintained below 50° C. The reaction mixture was stirred until the purple color disappeared. Brown precipitates were also formed.

The contents of the reaction vessel were filtered. The brown solid was washed with 1.0 M NaOH (100 ml×3). The filtrate was almost colorless. The filtrate was concentrated on a rotary evaporator to about ⅓ of the original volume and stirred with mechanical stirrer and cooled in ice bath. A 6 M HCl solution was added dropwise to reduce the pH of the mixture to about 2. White precipitates were formed during acidification. The precipitate was filtered, washed with de-ionized water, dried in the air and recrystallized in 2-propanol to give white solid of losartan 5-carboxylic acid (EXP-3174). Yield for the reaction was calculated at 78%.

The potassium salt of Losartan was also used for the reaction. The yield was 72%. ¹H NMR(DMSO-d6, 400 MHz): 7.64(d, 1H), 6.62(t, 1H), 7.55(t, 1H), 7.59(d, 1H), 7.07(d, 2H), 6.96(d, 2H), 5.62(s, 2H), 2.55(t, 2H), 1.51(q, 2H), 1.21(m, 2H), 0.85(t, 3H).

### Formulation

### Typical Pharmaceutical Compositions Containing the Compound of the Invention

### A: Dry Filled Capsules Containing 50 mg of Active Ingredient Per Capsule

Compound II (50 mg) can be reduced to a pharmaceutical useable powder and the lactose (149 mg) and magnesium stearate (1.0 mg) can then be passed through a No. 60 blotting cloth onto the powder. The combined ingredients can then be mixed for about 10 minutes and dispensed into a No. 1 dry gelatin capsule.

### B: Tablet

A typical tablet would contain compound II (25 mg), pregelatinized starch USP (82 mg), microcrystalline cellulose (82 mg) and magnesium stearate (1 mg).

### C: Suppository Formulation

A typical suppository formulations for rectal administration can contain compound II (1-25 mg), butylated hydroxyanisole (0.08-1.0 mg), disodium calcium edentate (0.25-0.5 mg), and polyethylene glycol (775-1600 mg). Other suppository formulation can be made by substituting, for example, butylated hydroxytoluene (0.04-0.08 mg) for the disodium calcium edentate and a hydrogenated vegetable oil (675-1400 mg) such as suppocire L, Wecobee FS, Wecobee M, Witepsoles, and the like, for the polyethylene glycol. Further, these suppository formulations can also include another active ingredient such as another antihypertensive and/or a diuretic and/or an angiotensin converting enzyme and/or a calcium channel blocker in pharmaceutically effective amounts.

### D: Injection

A typical injectable formulation would contain Compound II (5.42 mg), sodium phosphate dibasic anhydrous (11.4 mg), benzyl alcohol (0.01 ml) and water for injection (1.0 mg). Such an injectable formulation can also include a pharmaceutically effective amount of another active ingredient such as another antihypertensive and/or a diuretic and/or an angiotensin converting enzyme and/or a calcium channel blocker in pharmaceutically effective amounts.

1. A method of synthesizing losartan 5-carboxylic acid or a pharmaceutically acceptable salt thereof, comprising
combining water, potassium permanganate, and at least one polar inert organic solvent to form a first reaction mixture,
filtering the first reaction mixture to remove solids,
adding losartan to the filtered first reaction mixture to form a second reaction mixture,
adding formaldehyde to the second reaction mixture to form a first precipitate,
filtering the second reaction mixture to remove the first precipitate,
acidifying the filtered second reaction mixture to form a second precipitate,

2. The method of claim 1 wherein the losartan is selected from the group consisting of Compound I, an organic salt of Compound I and an inorganic salt of Compound I.

2. The method of claim 1 wherein the at least one polar inert organic solvent is selected from the group consisting of acetone, acetonitrile, THF, DMF, pyridine, and dipyridine.

4. The method of claim 1 wherein the second reaction mixture is maintained at temperature of −10 to 110° C.

5. The method of claim 4 wherein the second reaction mixture is maintained at temperature of 20° C. to 60° C.

6. The method of claim 1 further comprising
filtering the second precipitate,
washing the filtered second precipitate,
drying the filtered second precipitate, and
recrystallizing the dried filtered second precipitate in 2-propanol.

7. Losartan 5-carboxylic acid or a pharmaceutically acceptable salt thereof made by the method of claim 1.

8. A pharmaceutical formulation for the treatment of hypertension comprising a pharmaceutically acceptable carrier and an effective amount of the losartan 5-carboxylic acid or pharmaceutically acceptable salt thereof of claim 8.
